(19) United States
(12) Patent Application Publication
(10) Pub. No.: US 2021/0283287 A1
(43) Pub. Date: Sep. 16, 2021

(54) PORTABLE ELECTRONIC DEVICE SANITIZING CASE

(71) Applicants: Glenn D. Goodman, Springfield, MA (US); Brian Axler, Springfield, MA (US); Gary Goodman, Springfield, MA (US)

(72) Inventors: Glenn D. Goodman, Springfield, MA (US); Brian Axler, Springfield, MA (US); Gary Goodman, Springfield, MA (US)

(21) Appl. No.: 17/199,219

(22) Filed: Mar. 11, 2021

Related U.S. Application Data
(60) Provisional application No. 63/028,235, filed on May 21, 2020.
(60) Provisional application No. 63/017,330, filed on Apr. 29, 2020.
(60) Provisional application No. 62/988,710, filed on Mar. 12, 2020.

(51) Int. Cl. A61L 2/10; A45C 11/00

(52) U.S. Cl. CPC A61L 2/10; A45C 11/00; A45C 2011/002; A61L 2202/16; A61L 2202/12; A61L 2202/14

(57) ABSTRACT

A sanitizing case for electronic devices such as a portable listening device or mobile device. The sanitizing case comprises at least one UVC light source that exposes the electronic device in the case for a suitable period of time to destroy microorganisms. The case may also comprise a power switch to control the at least one UVC light source. The case may further include a power source to power the light source, as well as charge the electronic device. The invention provides a method for sanitizing electronic devices by exposing the device to UVC light for a predetermined period of time. The invention also provides a program or a mobile application that is capable of turning on and off the light source and setting the time of exposure.

### CROSS-REFERENCE TO RELATED APPLICATIONS

This patent application claims the benefit of U.S. Provisional Patent Application No. Patent Application No. 62/988,710 filed on Mar. 12, 2020, U.S. Provisional Patent Application No. 63/017,330 filed on Apr. 29, 2020 and U.S. Provisional Patent Application No. 63/028,235 filed on May 21, 2020, all of which are incorporated herein by reference.

### TECHNICAL FIELD OF THE INVENTION

The invention relates in general to mobile device cases and, specifically, to invertible device covers capable of sanitizing electronic devices like tablets, smartphones, ear buds or laptops.

### BACKGROUND

The use of mobile electronic computing devices, including smartphones, tablets, laptops, ear buds, and such continues to increase at high levels. It is estimated that more than 75% of American population use a smart phone. Further, electronic devices have also made their way into many professional environments, for example restaurants where tablets and smartphones are used to take orders. These electronic devices offer tremendous convenience as they are always in the user's vicinity, and their increasing computing power coupled with a vast number of applications allows them to be used in nearly all situations. Additional electronic devices such as ear buds also find significant use owing to the added convenience to common activities such as communication or watching videos or listening to music.

The increasing use of electronic devices is a cause for concern because of the ability of microorganisms, such as virus and bacteria, to accumulate and transfer through these devices. Given the fact that most of these devices utilize a haptic responsive system, coupled with the fact that user hands are in constant contact with a number of surfaces that aren't necessarily sterile, it is only natural to expect that the surfaces of electronics devices become a hotspot for such microorganisms. Further, with more virile species of viruses evolving that can transmit through air, these surfaces become targets for accumulation of such microorganisms. Thus, the likelihood of anyone to be infected by a microorganism is very high, and this condition can be exacerbated for the “vulnerable” population, such as children, those with compromised immune systems, aged members with comorbidities, etc.

Current methods for cleaning surfaces of electronics surfaces are rather inconvenient, and not portable. It generally requires cleaning chemicals, and the processes are quite time consuming. Further, if not used properly, such chemicals can affect the device functionality, and may also be harmful to humans, especially the vulnerable population.

Accordingly, there is a need for a method and a device that is capable of effectively and conveniently cleaning an electronic device without adding additional opportunities for transferring bacteria, which is also portable so it can be used anywhere.

### BRIEF DESCRIPTION OF THE INVENTION

In one aspect, the invention provides a portable listening device case. The portable listening device case comprises a housing having a receiving area configured to receive the portable listening device; a lid attached to the housing and operable between a closed position where the lid conceals the portable listening device within the case such that no light is allowed to penetrate the case and an open position that allows a user to remove the portable listening device from the receiving area; and at least one light source provided on at least one of the housing or the lid, or both.

In another aspect, the invention provides a mobile device case comprising a housing having a receiving area configured to receive the mobile device; a lid attached to the housing and operable between a closed position where the lid conceals the mobile device within the case such that no light is allowed to penetrate the case and an open position that allows a user to remove the portable listening device from the receiving area; and at least one light source provided on at least one of the housing or the lid, or both.

In yet another aspect, the invention provides a method for sanitizing an electronic device the method comprising providing a case for sanitizing the electronic device, wherein the case comprises a housing having a receiving area configured to receive the electronic device, a lid attached to the housing and operable between a closed position where the lid conceals the electronic device within the case such that no light is allowed to penetrate the case and an open position that allows a user to remove the electronic device from the receiving area, and at least one light source capable of generating light having wavelengths that ranges from about 185 nanometers to about 290 nanometers, wherein the light source is provided on at least one of the housing or the lid, or both; exposing the electronic device to the light for a predetermined period of time by turning the light source on or off.

In a further aspect, the invention provides a mobile application for sanitizing an electronic device comprising instructions to execute steps of the method as described herein.

### BRIEF DESCRIPTION OF DRAWINGS

These and other features, aspects, and advantages of the present invention will become better understood when the following detailed description is read with reference to the accompanying drawings in which like characters represent like parts throughout the drawings, wherein:

FIG. 1 shows one embodiment of the portable listening device case of the invention.

FIG. 2 shows another embodiment of the portable listening device case of the invention.

FIG. 3A is a back view of the mobile device case of the invention.

FIG. 3B is a top view of the mobile device case of the invention.

### DETAILED DESCRIPTION

The definitions provided herein are to facilitate understanding of certain terms used frequently herein and are not meant to limit the scope of the present disclosure.

As used in this specification and the appended claims, the singular forms “a”, “an”, and “the” encompass embodiments having plural referents, unless the content clearly dictates otherwise.

Unless otherwise indicated, all numbers expressing feature sizes, amounts, and physical properties used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings disclosed herein.

As used in this specification and the appended claims, the term “or” is generally employed in its sense including “and/or” unless the content clearly dictates otherwise.

As used herein, the phrase “UV-C light” means a range of electromagnetic radiation having a wavelength ranging from about one hundred nanometers to about two hundred eighty nanometers. It is known that UV-C light is up to 99.9% effective in the sanitization of microorganisms and biological pollutants/pathogens such as viruses, bacteria, mold dust mites, fungi, spores and flea eggs. Without being bound to any theory, it is hypothesized that UV-C light induces changes in the structure of DNA leading to the production and accumulation of cyclobutane pyrimidine dimers (CPDs), in turn distorting the DNA molecule, causing cellular damage. Thus, UV-C light has found tremendous use for sanitizing and disinfecting surfaces in various industries including healthcare (hospital, wound disinfection and healing), food processing, research laboratories, air purification systems and water purification applications. Further, when the power of the UV light in that wavelength range is less than about 15 Watts, it is known to be generally harmless to the human body.

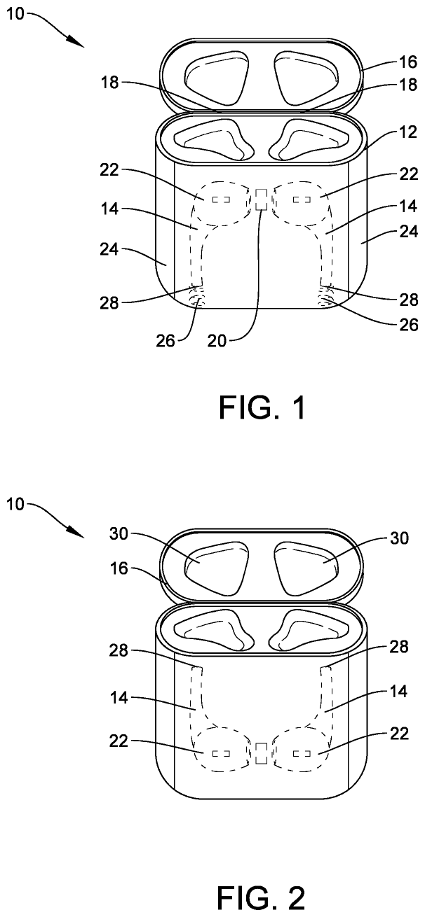

As noted herein, in one aspect, the invention provides a portable listening device case that can sanitize the listening device. Turning to the drawings, FIG. 1 depicts one embodiment of a portable listening device case of the invention, generally represented by numeral 10. In this embodiment, the case includes a housing 12 capable of receiving the portable listening device 14 and a lid 16 attached to the housing operable between an open and a closed position. Typical attachment means between the housing 12 and the lid 16 include hinges 18. However, the lid 16 may be attached to the housing 12 through other means, such as for example, wherein the lid can be completely removed from the housing. In a closed position, the lid 16 is configured to provide a sealed environment for the portable listening device 14. The seal may be achieved using several means known to one of obvious skill in the art, and may include, for example, but not limited to, a magnetic seal, a clasp, a clip, interlocking fibers such as hook-and-loop fasteners, other known fasteners, and the like, and combinations thereof.

The portable listening device case 10 includes at least one light source 20 that emits UVC radiation having power within the safe range as described herein. One skilled in the art will immediately understand that it is advantageously located close to the ear buds 22 as it comes into contact with the ear canal and is most susceptible to transmission of pathogens. Thus, when the portable listening device 14 is exposed to the UVC radiation for a period of time, all microorganisms and pathogens can be destroyed rendering the device safe for use without fear of further contamination. The at least one light source 20 could be a light emitting diode (LED) in one exemplary embodiment. As already described, the lid 16 is configured to provide a seal such that the light is not allowed to escape the confines of the case to maximize efficiency.

The portable listening device case 10 may also advantageously include a power source 24 with a suitable capacity that will provide power to the light source. The power source can also be used to charge the portable listening device. In FIG. 1, the power source is shown to be present on either sides of the housing 12, however, one skilled in the art will understand that it can be located at any suitable location. Further, the housing 12, the lid 16, or both may also comprise a solar panel that can provide power to the portable listening device, the at least one source, or both. The solar panel can be a supplemental power source or the only power source. The solar panel is located on the outside of the housing or the lid or both such that it can receive sunlight or other suitable radiation to charge the panel.

In the embodiment shown in FIG. 1, the case further includes a pair of springs 26 at a distal end from the lid. The springs are made of a conducting material and are connected to the power source 24. Thus, when the charging points of the portable listening device 28 come in contact with the springs 26, it can begin charging the portable listening device. The springs may further include press locks that can hold the portable listening device 14 in place while charging and when pressed again, the press lock will release the portable listening device.

In some embodiments, either the lid 16 or the springs 26 or both of them may be connected to the light source. In this manner, whenever the springs 26 are in a locked position or the lid 16 is in a closed position, or both, the light source 20 is automatically turned on. Additionally, the light source 20 is configured to be turned off whenever the springs are in the unlocked position and/or the lid 16 is in an open position. In further embodiments, the portable listening device case of the invention includes a power switch (not shown in FIG. 1) for the light source 20 that can be used to control the light source manually. The light source 20 may also be configured to automatically turn off after a predetermined period of time to save power.

FIG. 2 shows an alternate embodiment of the portable listening device case 10 of the invention, wherein it is configured to receive the portable listening device 14 headfirst. The charging points of the portable listening device 28 which comprises the conducting point for charging faces towards the lid. Thus, in this embodiment, the lid comprises points 30 connected to the power source, which when it comes in contact with the conducting point of the portable listening device 28 will charge it. Consequently, when the lid 16 is in a closed position, the charging occurs. As a further consequence, the light source is situated adjacent to the ear buds 22 of the portable listening device.

In some embodiments, the portable listening device case 10 further comprises a second light source (not shown in Fig) that can be used to detect the presence of germs. The second light source may be configured to provide light having a wavelength ranging from about 320 nm to about 370 nm. The power control for this second light source is distinct from the UVC light source and may be powered by a separate switch or through a program or a mobile application.

While the invention is described using the example of portable listening device such as AirPods® made available from Apple Corporation and their equivalents from other manufacturers, the case can also be used for other headsets such as wired headsets and Bluetooth headsets, wherein the case is configured to receive the ear buds portion to sanitize it. In such instances, some features such as charge points may not be necessary, whereas an external cable having USB, mini-USB, USB-C and the like connectors can be provided that will allow charging as necessary. One skilled in the art can arrive at the design of such a case without undue experimentation and hence, all such variations are also contemplated to be within the scope of the invention.

As already noted herein, in another aspect, the invention provides a mobile device case that can be used for sanitizing the mobile device. The invention is applicable to any type of mobile device and manufacturer and is not limited to cellular and smart phones but may be sized and configured for any device such as a portable tablet, personal digital assistant, portable computer, any electronic device and the like. The case of the invention may be adapted to protect the mobile device from impact, damage, scratches, etc using materials and constructions well known in the art such as rubber, plastic, polymer, metal and the like designed to protect edges, visual displays and built-in cameras while providing access to audio inputs/outputs, visual displays, control switches and the like. The case may also be configured to receive the mobile device in any direction—face up or face down—depending on the use situation. Further, the case is also designed to provide a snug fit for specific devices by configuring its size and shape to the respective device. For example, a specific case is designed for iPhone 11 and another for a Samsung Galaxy S20 Model, and so on. Such design of specific cases allows for some of the features and components of the device to be accessed while the device itself is being sanitized. For example, a specifically designed sanitizing case for Samsung Galaxy S20 will have suitable openings for speakers and cameras, thus allowing access to these components while still being sanitized.

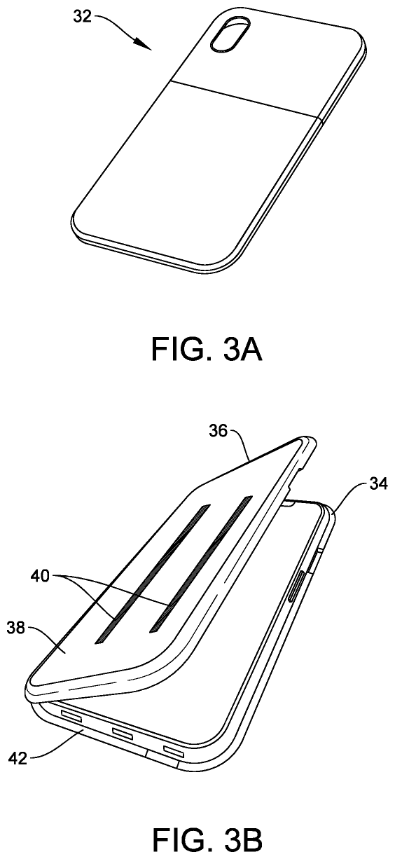

FIG. 3 shows one embodiment of the mobile device case 32. FIG. 3A is a back view of the mobile device cover of the invention, while FIG. 3B is a top view. The mobile device case 32 of the invention includes a housing 34 attached to a lid 36 operable between a closed position and an open position. In this exemplary embodiment, the housing is attached to the lid 36 through a hinge 38, however other means are also known in the art and are contemplated to be within the scope of the invention. For example, the lid can be completely detachable in one embodiment. Further, the lid 36 and housing 34 are configured to provide a total seal for the mobile device through suitable means, which may include, for example, but not limited to, a magnetic seal, a clasp, a snap-fit lock, a clip, fasteners such as hook-and-loop fasteners, and the like, and combinations thereof.

The mobile device case of the invention 32 further includes at least one light source 40 capable of emitting UVC radiation. Useful light sources in the invention include LED bulbs. The light sources 40 may be positioned at appropriate locations on the case to maximize exposure of the mobile device to the UVC radiation.

The mobile device case 32 may further include a power source receptacle (not shown in Fig) capable of receiving a suitable external power source such as a rechargeable battery. Alternately, a power source such as a battery pack may be provided at a suitable location on the mobile device case, one exemplary embodiment for the location of the battery pack 42 is shown in FIG. 3B. The power source may be used to charge the mobile device battery (not shown in FIG. 3A) as well as provide power to the at least one light source 40. Alternately, the mobile device case may include a solar panel (not shown in FIG. 3A) instead of, or in addition to the battery pack 42 as the power source. The solar panel is located on the outside of the housing or the lid such that it can receive sunlight or other suitable radiation to charge the panel.

The mobile device case may also comprise a power button (not shown in Fig) for the at least one light source 40, which would allow its manual control. Also, the light source may be connected to the lid 36 and/or the housing 34 such that it is configured to turn on when in the closed position, turn off when in the open position, or both.

In some embodiments, the mobile device case 32 includes a second light source (not shown in Fig) providing light having wavelength ranging from about 320 nm to about 370 nm to detect the presence of germs. The power control for this second light source is distinct from the UVC light source (not shown in Fig), and may be powered by a separate switch or through a program or a mobile application.

The mobile device case 32 can be used to sanitize the back and front side of the mobile device as the case is configured to receive the mobile device in face up or face down position. Further, the built-in camera of the mobile device can also be sanitized by appropriately exposing the UVC radiation on the camera. Alternately, a separate light source can be distinctly positioned at the location of the camera on the housing or the lid, as the case may be, to separately sanitize the camera.

Using the cases of the invention, the portable listening device and the mobile device can be sanitized in a facile manner. With the additional advantage of the cases being portable, the sanitization of the devices can take place at any location. It is also known that for sanitization to occur, the exposure to UVC light needs to occur for only a certain period of time, which can be preset for the light sources.

Thus, in a further aspect, the invention provides a method for sanitizing electronic devices. The method involves placing an electronic device in a sanitizing case wherein the sanitizing case is as described herein, and exposing the electronic device to the light from the at least one light source for a predetermined period of time.

Using the cases and the methods of the invention, electronic devices can be used in a safe manner in any location without fear of transmitting pathogens or being affected by pathogens transmitted through the use of electronic devices. This would also enable safe sharing of electronic devices with other users, which would be necessary during situations like gaming, studying or other social situations.

The method of the invention lends itself to being controlled suitably by a program or an application, which can be run from a suitable computing device such as a laptop or a mobile device. The application, for example, can be used to turn on or off at least one light source, or even set the time of exposure of the electronic device to the UVC radiation. Thus, in yet another aspect, the invention provides a mobile application for sanitizing the electronic devices. The mobile application can be used to turn on or off the at least one light source, turn on or off the second light source to detect presence of germs, set the time for which the at least one light source is on, perform analytics on the sanitizing activities by a user, aggregate multiple user statistics, and so on.

While only certain features of the invention have been illustrated and described herein, many modifications and changes will occur to those skilled in the art. It is, therefore, to be understood that the appended claims are intended to cover all such modifications and changes as fall within the true spirit of the invention.

We claim:

1. A case comprising:
a housing having a receiving area configured to receive a portable listening device;
a lid attached to the housing and operable between a closed position where the lid conceals the portable listening device within the case such that no light penetrates the case and an open position that allows a user to remove the portable listening device from the receiving area; and
at least one first light source attached to the housing and located within the housing.

2. The case of claim 1, further comprising a power button to control the at least one light source.

3. The case of claim 1, wherein the at least one first light source produces light having wavelength that ranges from about 185 nanometers to about 290 nanometers.

4. The case of claim 1, further comprising a second light source to detect germs attached to the housing and located within the housing.

5. The case of claim 4, wherein the second light source produces light having wavelength that ranges from about 320 nanometers to about 370 nanometers.

6. The case of claim 1, further comprising a power source.

7. The case of claim 6, wherein the power source is used to provide power to the portable listening device, the at least one first light source, or both independently.

8. The portable listening device case of claim 6, wherein the power source further comprises a solar panel located on at least one of outside of the housing, outside of the lid, or both

9. The case of claim 1, further comprising a closed lid sensor attached to the housing and located within the housing.

10. The case of claim 1, wherein the lid is detachable from the housing.

11. A case comprising:
a housing having a receiving area configured to receive a mobile device;
a lid attached to the housing and operable between a closed position where the lid conceals the mobile device within the case such that no light penetrates the case and an open position that allows a user to remove the mobile device from the receiving area; and
at least one first light source attached to the housing and located within the housing.

12. The case of claim 11, further comprising a power button to control the at least one light source.

13. The case of claim 11, wherein the at least one first light source produces light having wavelength that ranges from about 185 nanometers to about 290 nanometers.

14. The case of claim 11, further comprising a second light source to detect germs attached to the housing and located within the housing.

15. The case of claim 14, wherein the second light source produces light having wavelength that ranges from about 320 nanometers to about 370 nanometers.

16. The case of claim 11, further comprising a power source.

17. The case of claim 16, wherein the power source is used to provide power to at least one of the mobile device, the at least one first light source, or both independently.

18. The case of claim 16, wherein the power source comprises a solar panel located on at least one of outside of the housing, outside of the lid, or both

19. The case of claim 11, further comprising a closed lid sensor attached to the housing and located within the housing.

20. The case of claim 11, wherein the case further comprises a camera lens sanitizer located on the housing and configured to be aligned with the camera.

21. The case of claim 11, wherein the lid is detachable from the housing.

22. A method comprising:
placing an electronic device in a sanitizing case wherein the sanitizing case comprises:
a housing having a receiving area configured to receive the electronic device,
a lid attached to the housing and operable between a closed position where the lid conceals the electronic device within the case such that no light penetrates the case and an open position that allows a user to remove the electronic device from the receiving area, and
at least one first light source capable of generating light having wavelength that ranges from about 185 nanometers to about 290 nanometers, wherein the light source is attached to the housing and located within the housing;
exposing the electronic device to the light from the at least one light source for a predetermined period of time.

23. The method of claim 22, wherein the sanitizing case further comprises a power button located on the case.

24. The method of claim 23, further comprising turning off the at least one light source at the end of the predetermined period of time using the power button.

25. The method of claim 22, wherein the electronic device is a mobile device, or a portable listening device.
